(19) United States
(12) Patent Application Publication
(10) Pub. No.: US 2010/0125331 A1
(43) Pub. Date: May 20, 2010

(54) ASPHERIC INTRAOCULAR LENS WITH IMPROVED CONTROL OF ABERRATIONS

(76) Inventor: Michael J. Simpson, Arlington, TX (US)

Correspondence Address: ALCON, IP LEGAL, TB4-8, 6201 SOUTH FREEWAY, FORT WORTH, TX, 76134, US

(21) Appl. No.: 12/620,227

(22) Filed: Nov. 17, 2009

Related U.S. Application Data
(60) Provisional application No. 61/116,180, filed on Nov. 19, 2008.

(51) Int. Cl. A61F 2/16; A61B 3/10

(52) U.S. Cl. 623/6.11; 351/177; 351/246

(57) ABSTRACT

A method of designing an intraocular lens (IOL) of a selected power includes determining a power-specific axial separation parameter for the selected power. The method also includes selecting at least one aberration correction for the IOL. The method further includes designing the IOL based on the power-specific axial separation parameter to produce the selected aberration correction.

### RELATED APPLICATIONS

This application claims priority to U.S. Patent Application No. 61/116,180 filed Nov. 19, 2008.

### SUMMARY

In particular embodiments of the present invention, a method of designing an intraocular lens (IOL) of a selected power includes determining a power-specific axial separation parameter for the selected power. The method also includes selecting at least one aberration correction for the IOL. The method further includes designing the IOL based on the power-specific axial separation parameter to produce the selected aberration correction. In particular embodiments of the present invention, a method of manufacturing an intraocular lens (IOL) of a selected power includes determining a power-specific axial separation parameter for the selected power, selecting at least one aberration correction for the IOL, designing the IOL based on the power-specific axial separation parameter to produce the selected aberration correction, and manufacturing the IOL.

Further understanding of various aspects of the invention can be obtained by reference to the following detailed description in conjunction with the drawings, which are discussed briefly below.

### BRIEF DESCRIPTION OF THE DRAWINGS

FIG. 1 schematically depicts a model of an intraocular lens (IOL) within an eye according to a particular embodiment of the present invention;

FIG. 2 is a table tabulating an average anterior chamber depth (ACD) for groups of patients, in which each group of patients had a particular power of IOL implanted; and

FIG. 3 is a flow chart illustrating a method for designing an IOL according to a particular embodiment of the present invention.

### DETAILED DESCRIPTION

Particular embodiments of the present invention provide an intraocular lens (IOL) designed to correct aberration using a calculated anterior chamber depth that varies with IOL power. Such embodiments may advantageously improve the image contrast of the IOL resulting in improved vision for the patient. The term “intraocular lens” and its abbreviation “IOL” are used herein interchangeably to describe lenses that are implanted into the interior of the eye to either replace the eye's natural lens or to otherwise augment vision regardless of whether or not the natural lens is removed. Intracorneal lenses and phakic intraocular lenses are examples of lenses that may be implanted into the eye without removal of the natural lens.

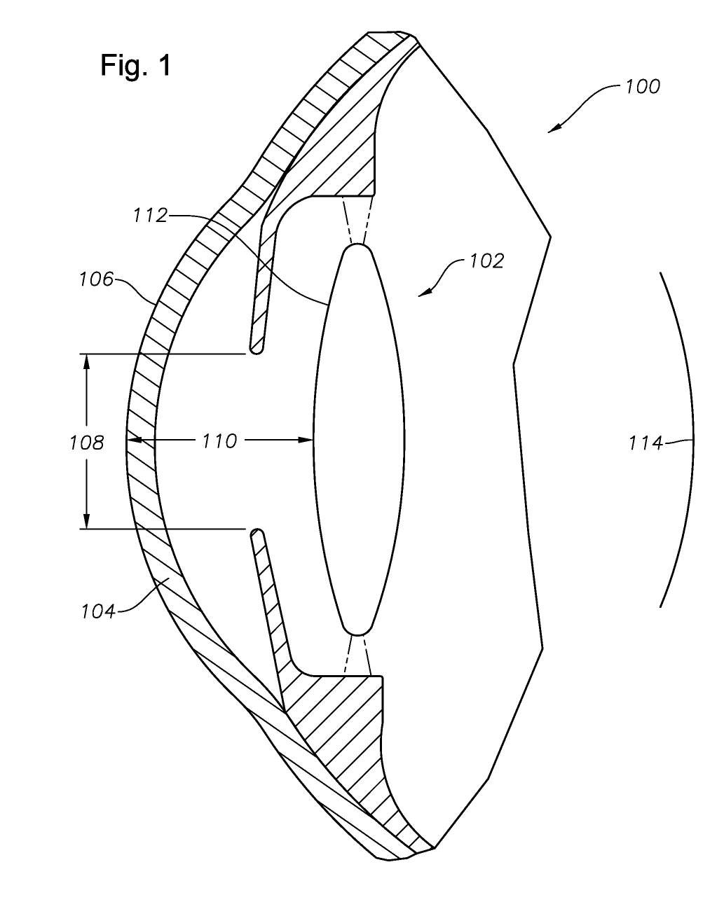

FIG. 1 schematically illustrates a model 100 of an IOL 102 within an eye. The model 100 includes a cornea 104 having an anterior surface 106. A pupil diameter 108 allowing light to travel to the IOL 102 may also be included in the model 100. The relative position of the IOL 102 and the cornea 104 may be quantified by an anterior chamber depth (ACD) 110 describing an axial distance between the anterior surface 106 of the cornea 104 and an anterior surface 112 of the IOL 102. Although the following description will be explained in terms of ACD, it should be understood that any parameter corresponding to a relative axial position between the cornea 104, IOL 102, and retina 114 (hereinafter referred to as an “axial separation parameter”), including ACD, may be used in any of the embodiments of the present invention described herein. In particular, the axial separation parameter may be back-calculated from measurements of eye length, corneal power, corneal asphericity, implanted IOL power, and postoperative refractive error to determine relative position for purposes of the model 100. In the model 100, the ACD 110 is selected to approximate the relative axial position of the IOL 102 when actually implanted in a living eye. The model 100 is used to determine how light rays will converge on the retina 114 to produce visible images.

Previous techniques for modeling IOLs have determined the ACD based on physical approximations of where the IOL would be disposed in a typical patient. An IOL is held in a particular position in the eye by haptics that contact particular anatomical features in the eye. For example, in the case of a foldable IOL used to replace the natural lens of an eye (also known as an “aphakic IOL”), the IOL can be held in place within the capsular bag. Similarly, in the case of an IOL that works in conjunction with the natural lens (also known as a “phakic IOL”), the haptics might contact the angle of the eye to hold the IOL in place. Because there may be variations in the relative position of these anatomical features of the eye relative to the cornea, the actual ACD of the IOL as implanted in the eye can vary from patient to patient. In designing IOLs, previous techniques attempted to develop a “best fit” for the ACD by determining an average over a large number of patients of the relative position of the cornea to these anatomical features that hold the IOL. A nominal value for the ACD based on such anatomical variations would typically be around 4.5 mm for an ordinary patient population.

One difficulty faced by IOLs designed according to such a methodology is that IOLs are often designed to control aberration through the use of aspheric surfaces, toric surfaces, diffractive structures, and the like. Such controlled aberration is particularly desirable to produce a desired depth of field for vision and to reduce or eliminate visible phenomena such as blurring, glares, halos, and the like. However, aberration corrections are quite sensitive to the axial position of the IOL as represented by the ACD. In real patients, the actual axial position of the IOL can range from as low as 3.5 mm to as high as 7.0 mm. These anatomical variations between patients can cause significant variation in the convergence of light at the retina produced by the IOL, which can cause significant variation in lens performance from patient to patient.

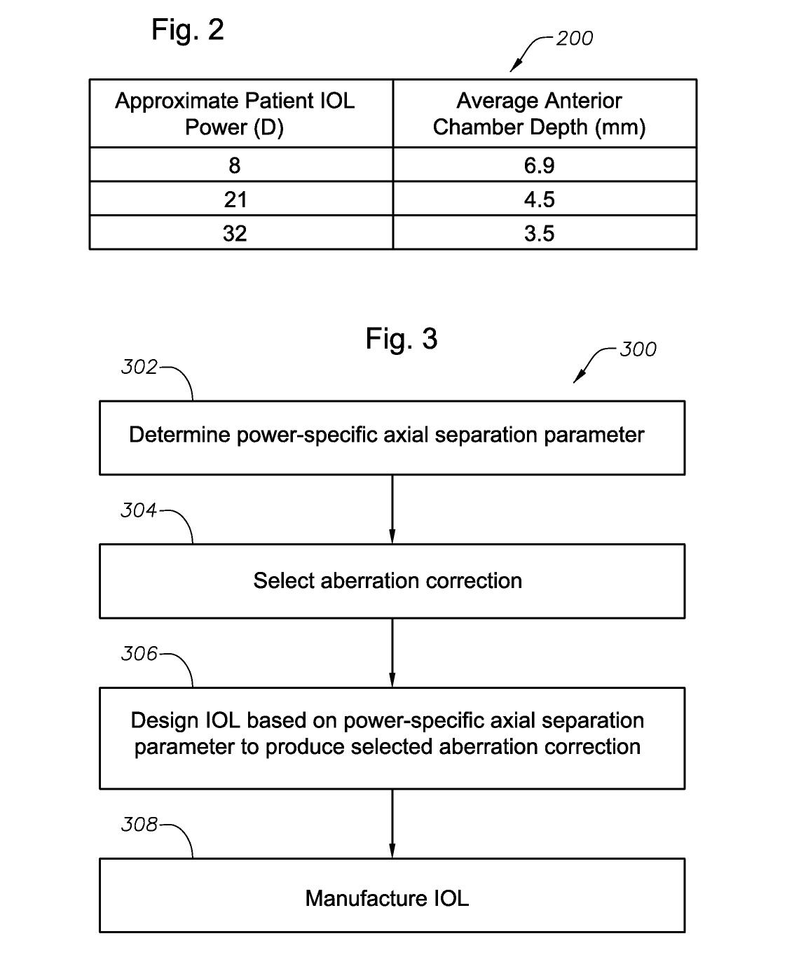

Techniques for designing an IOL according to particular embodiments of the present invention employ a modified ACD that also takes into account the power of the IOL. These techniques exploit a phenomenon that had not previously been remarked in IOL design methodologies, which is that patients requiring a higher power IOL frequently have a lower ACD than those requiring lower power IOLs. The table 200 in FIG. 2, which illustrates this phenomenon, tabulates results for a particular study of patient groups using different powers of ACRYSOF® IOLs, showing an average ACD for each patient group. The groups are collections of patients with similar implanted IOL powers, with the nominal IOL power for the group being an average of the various IOL powers within that group. Consequently, higher power IOL lenses can be designed for aberration control based on a lower (and therefore more accurate) ACD value determined for the particular lens power. For example, an IOL with a power of at least 25 D could have an ACD value less than 4 mm. Similarly, an IOL with a power less than 15 D could have an ACD value greater than 5.5 mm.

FIG. 3 is a flow chart 300 showing a method for designing and/or manufacturing an IOL according to a particular embodiment of the present invention. At step 302, a power-specific axial separation parameter is determined for an IOL. The power-specific axial separation parameter may be, for example, an average ACD value for a patient population in which IOLs of that power have been implanted. It may also be calculated for patient populations with similar IOL powers that also have similar axial lengths or corneal powers to each other. In another example, the power-specific value may be interpolated from average ACD values for different IOL powers. In general, the determination of a power-specific value involves any measurement of an actual axial separation parameter that is specifically associated with an IOL having a specific power and that is subsequently used to calculate a theoretical ACD based on IOL power. At step 304, a desired aberration correction is selected. This may be determined, for example, by specifying the desired image contrast, at the macula of the retina, or by specifying other parameters such as the level of spherical aberration, the correction or control of astigmatism, the creation of a desired depth of field, or to produce any other desired modification of aberration. Furthermore, the desired aberration correction may include multiple different types of aberration correction in combination. At step 306, the IOL is designed based on the power-specific axial separation parameter to produce the selected aberration correction. The IOL may then be manufactured at step 308.

While the foregoing discussion has specifically addressed using the connection between an axial separation parameter and the power of an implanted IOL to determine a power-specific axial separation parameter, it should also be noted that the connection between the axial separation parameter and IOL power can also be exploited in other ways. In particular, the relationship among optical properties of the cornea (e.g., asphericity, power), eye length, and IOL power may be used to determine both the power and the axial separation parameter used in the eye model. Thus, it is possible to have a method for selecting an IOL that includes determining eye length and at least one optical property of a cornea, determining an IOL power and an aberration correction based on the eye length and the at least one optical property of the cornea, and implanting an IOL having the selected power and the aberration correction. Similarly, this concept could extend to a method for designing and/or manufacturing an IOL that includes selecting an eye length and at least one optical property of a cornea, determining an IOL power and an aberration correction based on the eye length and the at least one optical property of the cornea, and designing and/or manufacturing an IOL with the IOL power and the aberration correction. Such a method may include grouping patients by a combination of eye length and corneal properties so that, for example, an eye of shorter length but higher corneal power might use a lens with a power normally associated with a lesser corneal power in an average eye.

The present invention has been described by reference to certain preferred embodiments; however, it should be understood that it may be embodied in other specific forms or variations thereof without departing from its essential characteristics. The embodiments described above are therefore considered to be illustrative in all respects and not restrictive, the scope of the invention being indicated by the appended claims.

1. A method of designing an intraocular lens (IOL) of a selected power, comprising:
determining a power-specific axial separation parameter for the selected power;
selecting at least one aberration correction for the IOL; and
designing the IOL based on the power-specific axial separation parameter to produce the selected aberration correction.

2. The method of claim 1, wherein the power-specific axial separation parameter is an anterior chamber depth (ACD).

3. The method of claim 2, wherein the selected power is at least 25 D and the anterior chamber depth is less than 4.0 mm.

4. The method of claim 2, wherein the selected power is less than 15 D and the anterior chamber depth is at least 5.5 mm.

5. The method of claim 1, wherein the IOL is an aphakic IOL.

6. The method of claim 1, wherein the IOL is a phakic IOL.

7. The method of claim 1, wherein the aberration correction is produced by an aspheric surface of the IOL.

8. The method of claim 1, wherein the aberration correction is produced by a tonic surface of the IOL.

9. The method of claim 1, wherein the aberration correction is determined based on a selected depth of field for the IOL.

10. A method of manufacturing an intraocular lens (IOL) of a selected power, comprising:
determining a power-specific axial separation parameter for the selected power;
selecting at least one aberration correction for the IOL;
designing the IOL based on the power-specific axial separation parameter to produce the selected aberration correction; and
manufacturing the IOL.

11. The method of claim 10, wherein the power-specific axial separation parameter is an anterior chamber depth (ACD).

12. The method of claim 11, wherein the selected power is at least 25 D and the anterior chamber depth is less than 4.0 mm.

13. The method of claim 11, wherein the selected power is less than 15 D and the anterior chamber depth is at least 5.5 mm.

14. The method of claim 10, wherein the IOL is an aphakic IOL.

15. The method of claim 10, wherein the IOL is a phakic IOL.

16. The method of claim 10, wherein the aberration correction is produced by an aspheric surface of the IOL.

17. The method of claim 10, wherein the aberration correction is produced by a tonic surface of the IOL.

18. The method of claim 10, wherein the aberration correction is determined based on a selected depth of field for the IOL.

19. A method of selecting an IOL for a patient, comprising:
determining an eye length and at least one optical property of a cornea for the patient;
determining an IOL power and an aberration correction based on the eye length and the at least one optical property of the cornea; and
implanting the IOL in the patient.
